(19) United States
(12) Patent Application Publication
(10) Pub. No.: US 2024/0082044 A1
(43) Pub. Date: Mar. 14, 2024

(54) WHEELCHAIR SECURABLE URINE COLLECTION SYSTEMS AND RELATED METHODS

(71) Applicant: PureWick Corporation, Covington, GA (US)

(72) Inventors: Hoang Nguyen, Lawrenceville, GA (US); Charles Shermer, Raleigh, NC (US); Katherine Van Orden, Decatur, GA (US); Camie L. Meller, Covington, GA (US); Eric Alan Fallows, Apex, NC (US); Eric Portante, Wake Forest, NC (US); Hersh Patel, Atlanta, GA (US); Melody Kuroda, Durham, NC (US); Melissa Young Joyner, Stone Mountain, GA (US); Dana Ahmad Abdelal, Marietta, GA (US); Vinayaka Kulkarni, Bangalore (IN); Audrey Earnshaw, Erie, CO (US); Patrick Hudson Chancy, Dunwoody, GA (US)

(21) Appl. No.: 18/260,122

(22) PCT Filed: Jan. 6, 2022
(86) PCT No.: PCT/US2022/011421

§ 371 Date: Jun. 30, 2023

Related U.S. Application Data
(60) Provisional application No. 63/134,631, filed on Jan. 7, 2021.

(51) Int. Cl. A61F 5/44; A61F 5/441; A61G 5/10

(52) U.S. Cl. CPC A61F 5/4404; A61F 5/4401; A61F 5/441; A61G 5/1002

(57) ABSTRACT

A portable urine collection system (200) may include a urine collection device, a conduit (217) in fluid communication with the urine collection device (212), a urine collection container (214) having an interior region, a pump (216), and a container support (202). The pump is in fluid communication with the urine collection container and is configured to pull a vacuum on the interior region of the urine collection container effective to draw urine from the urine collection device through the conduit into the urine collection container. The container support is configured to detachably secure to a wheelchair and support at least the urine collection container.

### CROSS-REFERENCE TO RELATED APPLICATIONS

This application claims priority to U.S. Provisional Patent Application No. 63/134,631 filed on Jan. 7, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to fluid collection devices and methods of using fluid collection devices. In an embodiment, a portable urine collection system is disclosed. The urine collection system includes a urine collection device configured to be positioned at least proximate to a urethra of a user. The urine collection system also includes a first conduit in fluid communication with the urine collection device and a urine collection container having an interior region. The urine collection system also includes a container support configured to detachably secure to a wheelchair and support at least the urine collection container.

In an embodiment, a method of assembling a portable urine collection system is disclosed. The method includes detachably securing a container support to a wheelchair. The method also includes mounting a urine collection container to the container support. The method also includes positioning a urine collection device proximate to a urethra of a user. The method also includes fluidly coupling the urine collection device to the urine collection container with a first conduit. The method also includes mounting a pump to the wheelchair with the pump in fluid communication with the urine collection container and configured to pull a vacuum on the interior region of the urine collection container effective to draw urine from the urine collection device through the first conduit into the urine collection container.

In an embodiment, a urine collection system includes a urine collection device configured to be positioned at least proximate to a urethra of a user, a urine collection container having an interior region, a nanofiber membrane positioned within the urine collection container, and a conduit in fluid communication with the urine collection device and the urine collection container.

In an embodiment, a method of collecting urine from a user is described. The method includes fluidly coupling a conduit to a urine collection container. The method also includes positioning a urine collection device proximate to a urethra of a user with the conduit providing fluid communication between the urine collection device and an interior region of the urine collection container. The method also includes absorbing urine with a urine absorption material positioned in the interior region of the urine collection container after the urine has been discharged by the user into the urine collection device and passed through the conduit into the interior region of the urine collection container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a portable urine collection system, according to an embodiment.

FIG. 2A is a rear isometric view of a portable urine collection system secured to a wheelchair, according to an embodiment.

FIG. 2B is the portable urine collection system of FIG. 2A without the wheelchair.

FIG. 2C is a belt of the portable urine collection system, according to an embodiment.

FIG. 3A is a side view of a portable urine collection system secured to a wheelchair, according to an embodiment.

FIG. 3B is a side view of the portable urine collection system of FIG. 3A without the wheelchair.

FIG. 3C is a front view of a urine collection container of the portable urine collection system of FIG. 3A, according to an embodiment.

FIG. 3D is a front view of a nanofiber membrane removed from the urine collection container of the portable urine collection system of FIG. 3A, according to an embodiment.

FIG. 3E is a front view of a nanofiber membrane of a portable urine collection system before urine has been absorbed by the nanofiber membrane, according to an embodiment.

FIG. 3F is a front view of the nanofiber membrane of FIG. 3E after urine has been absorbed by the nanofiber membrane, according to an embodiment.

FIG. 4A is a front right isometric view of a portable urine collection system mounted to a wheelchair, according to an embodiment.

FIG. 4B is a front left isometric view of the portable urine collection system of FIG. 4A mounted to a wheelchair with the pad removed.

FIG. 4C is a front isometric view of a portable urine collection system, according to an embodiment.

FIG. 4D is a front isometric view of a portable urine collection system, according to an embodiment.

FIG. 4E is a rear isometric view of a portable urine collection system, according to an embodiment.

FIG. 4F is a rear isometric view of a portable urine collection system, according to an embodiment.

FIG. 5 is a block diagram of a controller in the urine collection system of FIG. 3A, according to an embodiment.

FIG. 6 is a flow diagram of a method for assembling a portable urine collection system, according to an embodiment.

### DETAILED DESCRIPTION

Embodiments disclosed herein are related to wheelchair securable urine collection systems and related methods. Many users of urine collection devices are over 65 years old with limited mobility, often relying on wheelchairs as a primary mode of transportation. Many users also spend a significant amount of their day in a seated or supine position. Users and caregivers, then, are benefited from a urine collection system that may be both discrete and mobile, allowing users to use the urine collection system to collect urine both at home and on the go.

In at least one, some, or all of the embodiments described herein, a urine collection system is compact and includes a configuration resulting in the technical effect of allow the urine collection system to be secured or mounted to a wheelchair. At least one, some, or all of the embodiments of the urine collection systems described herein are mobile and discreet, providing the technical effect allowing a user to participate in social activities without alerting others to the incontinence of the user. For example, the urine collection systems may include a cover configured to hold at least the pump of a urine collection system therein, resulting in the technical effect of obscuring the pump from view outside the urine collection system. The urine collected in the urine collection systems described herein also may be stored in a urine collection container that is obscured from view outside the urine collection system and/or obscures the urine held in the urine collection container. In many embodiments, the urine collection system also includes an alert system configured to communicate with an electronic device, resulting in the technical effect of alerting a user or caregiver to empty the urine collection container when the urine level approaches a predetermined level, to change or recharge a battery, and/or adjust a vacuum or suction level of the pump in the urine collection system.

Many embodiments of urine collection systems described herein also may include an absorptive material positioned or positionable in a urine collection container. The absorptive material, such as a nanofiber membrane, may be configured to expand and/or form a structured hydrogel from the absorptive material as urine is absorbed by the absorptive material. Accordingly, many urine collection systems described herein may include urine solidification systems. The use of an absorptive material that may solidify to form a structured hydrogel results in the technical effect of preventing potential leaks from a urine collection bag and/or embarrassing noises emanating from the urine collection system as urine is collected. The absorptive material may be degradable, thus advantageous to conventional super-absorbent polymers used in disposable diapers, menstrual pads, and other sanitary napkins.

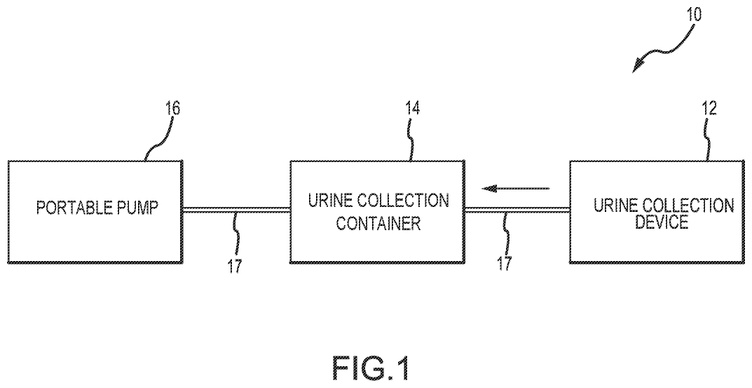

FIG. 1 is a block diagram of a fluid collection system 10, according to an embodiment. The fluid collection system 10 may be included in embodiments of fluid collection systems described herein. The system 10 includes a fluid (e.g., urine) collection device 12 (e.g., any of the fluid collection assemblies disclosed herein), a urine collection container 14, and a pump 16 (or vacuum source). The fluid collection device 10, the urine collection container 14, and the pump 16 may be fluidly coupled to each other via one or more conduits 17. For example, fluid collection device 10 may be operably coupled to one or more of the urine collection container 14 or the pump 16 via the conduit 17. In some embodiments, the pump 16 may be secured directly to the urine collection container 14. Fluid (e.g., urine or other bodily fluids) collected in the fluid collection device 10 may be removed from the fluid collection device 10 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the urine collection container 14. For example, the outlet of the conduit 17 may be disposed within or fluidly coupled to an interior region of the urine collection container 14 and an additional conduit 17 may extend from the urine collection container 14 to the pump 16. Accordingly, the pump 16 may apply suction to the fluid collection device 12 via the urine collection container 14. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle or cup (e.g., collection jar), or any other enclosed container for storing bodily fluid(s) such as urine. In some examples, the conduit 17 may extend from the fluid collection device 12 and attach to the urine collection container 14 at a first point therein. An additional conduit 17 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 or vacuum source may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

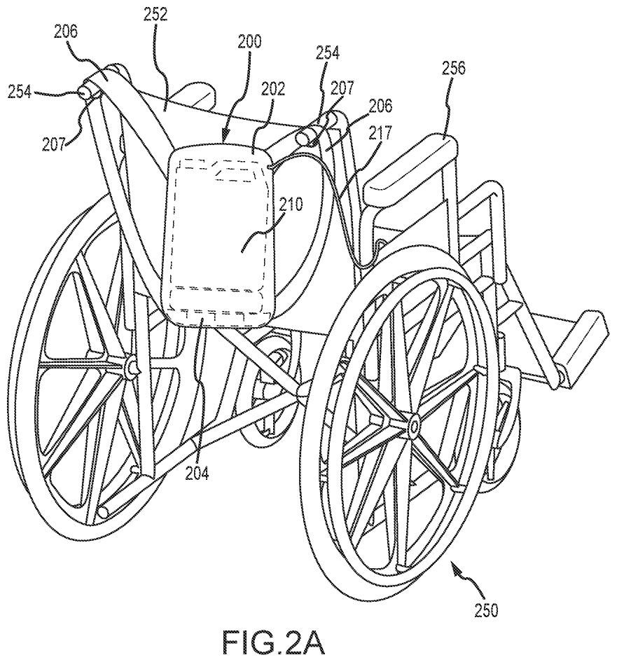

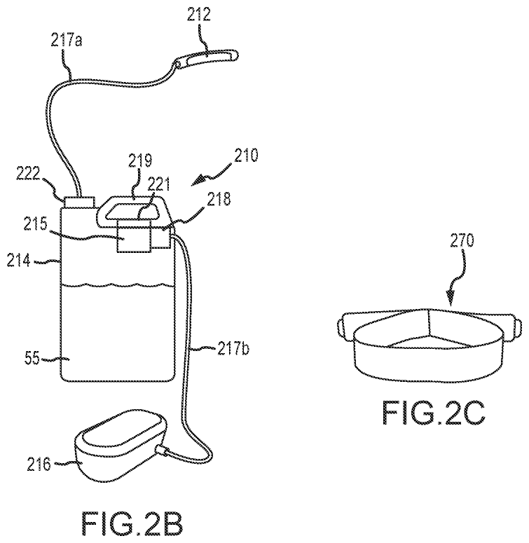

The configuration of at least one, some, or all of the embodiments of urine collection systems described herein provides the technical effect of allowing the urine collection systems to be worn by a user, positioned on a surface such as a table, and/or securable or mountable to a wheelchair. Turning to FIG. 2A, a urine collection system 200 is shown secured or mounted to a wheelchair 250. The urine collection system 200 also may be worn by a user and/or caregiver. Whether mounted to a wheelchair 250 or worn by a user, the configuration of the urine collection system 200 results in the technical effect of allowing a user to discretely use and/or transport the urine collection system 200 with the cover 202 that holds a urine storage system 210 therein. For example the cover 202 may be sized and dimensioned to hold at least a urine collection container 214, pump 216, and a sensor 215 (shown in FIG. 2B) therein.

The wheelchair 250 may include any of a number of different conventional wheelchairs, and may include a back 252, two handles 254, and two arms 256. The urine collection system 200 may include a container support 202 or cover that is configured to detachably secure, mount, or hang from the wheelchair 250 and support a storage system 210 of the urine collection system 200. In some embodiments, the container support 202 includes a pack or bag having one or more straps 206 configured to hang from or secure to one or more handles 254 of the wheelchair 250. The straps 206 may include one or more fasteners configured to adjustably secure the straps 206 to the wheelchair 250, such as at least one of buckles, clips, and/or hook and loop fastener materials. The straps 206 may each include a ring 207 sized and dimensioned to insert the handle 254 of the wheelchair 250 therethrough or other suitable device. The ring 207 may be positioned on an inner surface of the strap 206, as shown in FIG. 2B, or an outer surface of the strap 206 such that the strap 206 does not hang over the handle 254. The ring 207 may be movably secured or securable to the strap 206. The straps 206 may be dimensioned to allow a user or caregiver to adjust the straps 206 to move the container support 202 closer to the ground. By moving the container support 202 below a level of the seat of the wheelchair 250, the technical effect of pulling urine from the urine collection device 212 and into the urine collection container 214 with the assistance of primarily or entirely of gravity is achieved. In some embodiments, the container support 202 is configured to be worn as a backpack using the one or more straps 206.

The pack or bag of the container support 202 is sized and dimensioned to hold at least the urine collection container 214 therein. The pack of the container support 202 also may include a sleeve sized and dimensioned to hold the urine collection container 214 therein. The sleeve of the container support 202 may be insulated, such as with one or more foam members 204. The positioning and configuration of the one or more foam members 204 in the container support 202 result in the technical effect of reducing operational sounds of the urine collection system 200 (e.g. a pump 216, shown in FIG. 2B). In addition to abating sound, the one or more foam members may result in the technical effect of minimizing operational vibrations of the urine collection system 200. The one or more foam members 204 may be positioned underneath and/or around at least a portion of the pump 216 to minimize vibrations and sounds emanating from the pump 216. In some embodiments, the one or more foam members 204 may be positioned in one or more sleeves in an interior region of the container support 202. The pack of the container support 202 may include a reinforced base resulting in the technical effect of keeping the pack upright and preventing fluid ingress into the pump 216. In some embodiments, the pack of the container support 202 may include a heat sink positioned below the pump 216 to inhibit the pump 216 from overheating. The pack of the container support 202 may include one or more air vents on the side or front of the pack for pulling air into the pack.

Turning to FIG. 2B, which shows a urine collection system 200 removed from the container support 202, according to an embodiment. The urine collection system 200 may include a urine collection device 212, a conduit 217, a storage system 210, and a pump 216. The urine collection device 212 is configured to be positioned at least proximate to a urethra of a user. While the urine collection device 212 shown in FIG. 2B includes a female urine collection device, the urine collection device 212 may instead include a male urine collection device. PCT International Application No. PCT/US2019/029616, for example, describes various embodiments of both male and female fluid collection devices, the disclosure of which are incorporated in their entirety by this reference. Moreover, the urine collection device 212 may be interchangeable in the urine collection system 200 between different types, varieties, and sizes of male or female urine collection devices. Generally, the urine collection device 212 may include a surface sized to be positioned proximate or adjacent to the urethra and configured to wick urine or other fluids away from the user. Urine or other fluids may be wicked from the surface to a reservoir in the urine collection device 212.

The urine collection system 200 also includes a first conduit 217a in fluid communication with an interior region (e.g. reservoir) of the urine collection device 212 and an interior region of the urine collection container 214. The first conduit 217a may be positioned between the urine collection device 212 and the urine collection container 214. The urine collection system 200 also may include a second conduit 217b providing fluid communication between the pump 216 and the interior region of the urine collection container 214. In some embodiments, the pump 216 may be secured directly to the urine collection container 214, and the second conduit 217b may be absent from the urine collection system 200. The conduits 217a, 217b may include a flexible tube. In some embodiments, at least a portion of the first conduit 217a is substantially opaque, thereby inhibiting viewing of the urine within the first conduit 217a.

The urine collection system 200 also includes a storage system 210, according to an embodiment. The storage system 210 may include the urine collection container 214 having an interior region that stores urine 55 received from the urine collection device 212 via the first conduit 217a. The urine collection container 214 may be opaque or clear according to different embodiments and may include a generally rectangular front or rear profile. In some embodiments, the urine collection container 214 includes at least one of a handle 219 or a spout 222. Urine 55 collected in the urine collection container 214 may be emptied through the spout 222 after removing a cap or cover. The urine collection container 214 may be reusable and dishwasher safe, and may include a generally rigid material such as polycarbonate, plastic, rubber, metal, glass, combinations thereof, or any other suitable materials. The urine collection container 214 may be sized and dimensioned to fit within an insulated sleeve in the pack of the container support 202.

The storage system 210 also may include a filter 218, a controller 221, and a sensor 215. The sensor 215 is secured to the urine collection container 214 and configured to detect a property relating at least to a volume of the urine 55 in the interior region of the urine collection container 214. In some embodiments, the sensor 215 may be located or positioned at an inlet (either interior or exterior) of the urine collection container 214 for the conduit 217a. The sensor 215 may be positioned along and/or within the conduit 217a, according to an embodiment. In some embodiments, the sensor 215 includes an ultrasonic sensor, a laser sensor, or an ultraviolet (UV) sensor configured to provide a continuous or periodical feedback of the property relating at least to a volume of the urine 55 without the sensor touching the urine 55. The sensor 215 may include a non-contact fluid sensor such as a capacitive sensor, an inductive sensor, a gravimetric sensor, or a mechanical float. The sensor 215 may be secured to the urine collection container with at least a portion of the sensor 215 inside the interior region of the urine collection container 214. In some embodiments, the sensor 215 is positioned at a top of the urine collection container 214 and pointed downwards where the urine 55 collects in the interior region of the urine collection container 214.

In some embodiments, the sensor 215 includes a level transmitter configured to detect a level of the urine 55 in the urine collection container 214. The sensor 215 may include a laser level sensor that measures the distance between the laser transmitter of the sensor 215 to a surface of the urine 55 and back to a detector of the sensor 215. The elapsed time between transmitting the laser and detecting the reflected laser may be measured by the sensor 215 and a distance between the sensor 215 and the level of the urine 55 may be calculated by the sensor 215 or an associated controller 221. The laser may be transmitted through a clear window on the urine collection container 214 or at least the transmitter of the sensor 215 may be positioned within the urine collection container 214.

In some embodiments, the sensor 215 may include an ultrasonic level sensor that leverages the speed of sound to detect the property relating at least to a volume of the urine 55 in the urine collection container 214. For example, the sensor 215 may measure a distance between a transducer of the sensor 215 and a surface of the urine 55 in the urine collection container 214 using a time for the pulse to travel from the transducer to the surface of the urine 55 and back to the transducer of the sensor 215. The sensor 215 or a controller 221 associated with the sensor 215 may determine a volume of the urine 55 in the urine collection container 214 using the distance between the sensor 214 and the surface of the urine 55 in the urine collection container 214.

In some embodiments, the sensor 215 includes or is associated with an accelerometer providing the technical effect of determining when the sensor 215 should detect the level of the urine 55 in the urine collection container 214. The accelerometer is configured to detect a steady state of the urine 55 in the urine collection container 214 when the sensor 215 may transmit the laser or ultrasonic pulse for detecting a level of the urine 55 in the urine collection container 215. For example, the accelerometer may detect or indicate when the urine collection container 214 has been stationary for a predetermined or preselected period of time to steady the readings from the sensor 215 and improve accuracy of the readings from the sensor 215.

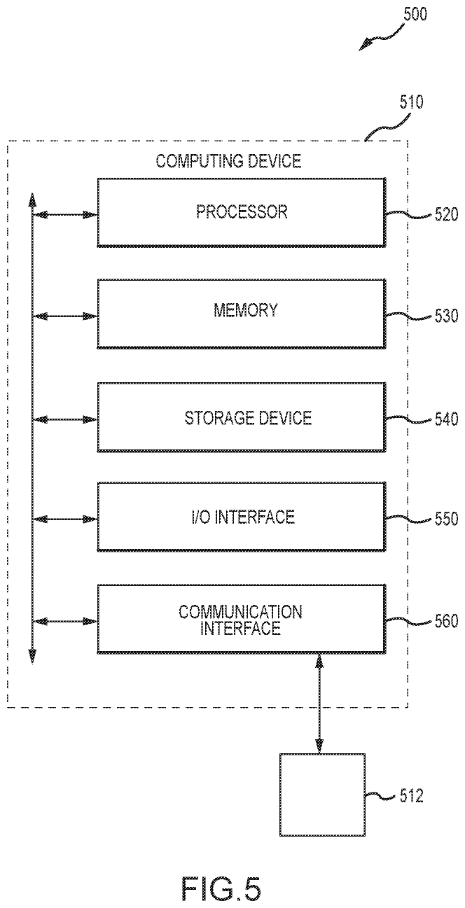

In some embodiments, the storage system 210 also includes the controller 221. The controller 221 may be configured to communicate with the sensor 215, such as a wired or wireless connection. In some embodiments, the sensor 215 may include the controller 221. The controller 221 may include a printed circuit board (PCB) equipped with erasable programmable read-only memory (EPROM) for memory of at least data collected by the sensor 215. The controller 221 may include a processor configured to calculate a level or volume of urine 55 in the urine collection container. The controller 221 may include a communication interface configured to send notifications or alerts to other electronic devices, such as a smartphone of the user or caregiver, an electronic device secured to the wheelchair 250 having a module and/or a display, and/or an electronic device of a healthcare system. For example, the communication interface may be configured to send notifications or alerts at a selected radio frequency, via BLUETOOH, or via WI-FI to another electronic device, such as a smartphone of the user or caregiver or an electronic device secured to the wheelchair 250. The controller 221 may be powered by an external or internal battery, such as a rechargeable battery. FIG. 5 provides additional details of a controller 500 that may include the controller 221.

In some embodiments, the controller 221 is configured to wirelessly transmit an alert to an electronic device of the user or a caregiver when the property relating at least to the volume of the urine detected by the sensor 215 indicates the volume of the urine 55 in the urine collection container 215 has reached or exceeded a predetermined volume. For example, based on data from the sensor 215, the controller 221 may wirelessly transmit an alert to an electronic device that the urine 55 in the urine collection device is a predetermined distance (such as about 2.5 cm) from the sensor 215, and emptying of the urine collection container 214 is recommended. In some embodiments, the controller 221 may wirelessly transmit alerts and selected frequencies, such as selected time and/or volume intervals. The controller 221 may wirelessly transmit an alert to the electronic device of the user or the caregiver when a battery powering at least one of the controller 221 or the pump 216 is low. The controller 221 may wirelessly transmit an alert to the electronic device of the user or the caregiver when replacement of a filter (such as the filter 218) of the urine collection system 200 is recommended.

The storage system 210 also may include the filter 218 resulting in the technical effect of neutralizing odor of the air being pulled from the interior region of the urine collection container 214 by the pump 216. In some embodiments, the filter 218 is positioned between at least a portion of the conduit 217b and a portion of the interior region of the urine collection container 214 such that air being pulled from the interior region of the urine collection container 214 is filtered before or as the air enters the conduit 217b. In some embodiments, the filter 218 is positioned on an exhaust vent on the pump 216. In some embodiments, a filter 218 is positioned both at the exhaust vent on the pump 216 and before air enters the conduit 217b. The filter 218 may include an odor absorbing filter and/or a hydrophobic filter configured to prevent or minimize fluid from the urine collection container 214 being pulled into the pump 216.

In some embodiments, the filter 218 may include an aromatherapy pack or an aromatherapy pack may be secured proximate to the exhaust vent of the pump resulting in the technical effect of producing a more pleasant smell. The filter may include baking soda or other composition that removes odor from the air and/or adds pleasant aroma to the air. In some embodiments, the filter 218 is absent and the pump 216 includes the aromatherapy pack removably secured or securable to the pump 216 proximate to the exhaust vent.

The pump 216 is in fluid communication with the interior region of the urine collection container 214 and is configured to pull a vacuum on the interior region of the urine collection container 214 effective to draw the urine from the urine collection device 212 through the first conduit 217a into the urine collection container 214. The pump 216 may be secured directly to the urine collection container 214, or the conduit 217b may fluidly couple the pump 216 with the interior region of the urine collection container.

The pump 216 may include one or more of a manual vacuum pump, and electric vacuum pump, a diaphragm pump, a centrifugal pump, a displacement pump, a magnetically driven pump, a peristaltic pump, or any pump configured to produce a vacuum. For example, the pump may include an air media diaphragm pump having a minimum pumping speed measured in ml/second. In some embodiments, the pump 216 includes a variable speed pump and/or a continuous pump. For example, the pump 216 may include a variable speed pump that operates at a low speed until the sensor 215 or other sensor detects urine passing through the conduit 217a into the urine collection container 214, when the pump 216 then adjusts to a higher speed to prevent wetting or pooling of urine at the urine collection device 212. In some embodiments, the urine collection system includes the sensor 215 positioned within the conduit 217a, at an inlet of the conduit 217a, or at an outlet of the conduit 217a, and the pump 216 is activated when the sensor 215 senses fluid to promote fluid through the conduit 217a into the urine collection container 214. The pump 216 may provide a vacuum or suction to remove fluid from the fluid collection device 212. In some examples, the pump 216 may be powered by one or more batteries. In some examples, the pump 216 may be sized and shaped to fit within the container support 202, such as within a sleeve within the container support 202. For example, the vacuum source 216 may include one or more miniaturized pumps or one or more micro pumps. The pump 216 may include one or more of a switch, a button, a plug, a remote, or any other device suitable to activate the pump 216.

Turning to FIG. 2C, the urine collection system 200 also may include one or more belts 270. The one or more belts 270 may be configured to secure the conduit 217a to the user and/or a portion of the wheelchair 250. The one or more belts 270 may include a fastener configured to allow the belt to adjust in size, such as an elastic material, hook and loop fastener material, a buckle, a clip, and so on.

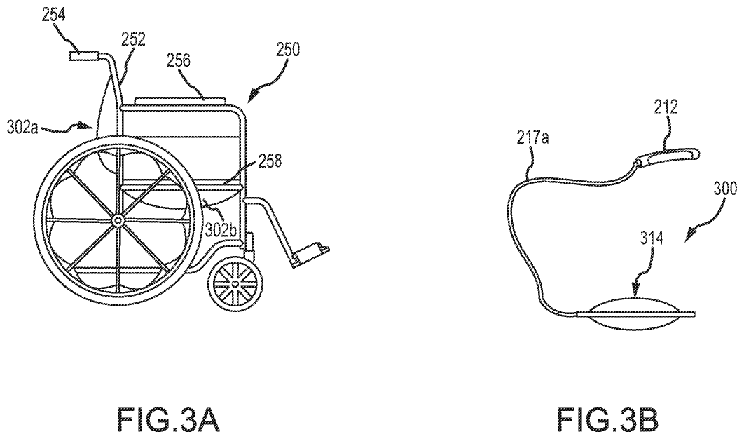

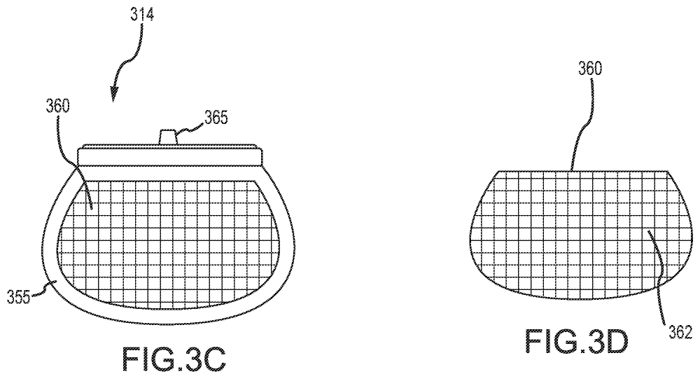

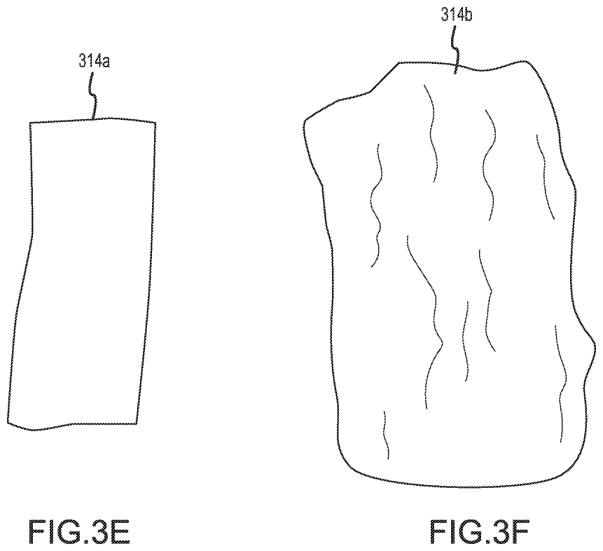

In some embodiments, the container support of a urine collection system may include a fabric sling configured to detachably secure to a wheelchair. Turning to FIG. 3A, the container support may include at least one of a fabric sling 302a configured to detachably secure to the back 252 of the wheelchair 250 or a fabric sling 302b configured to detachably secure to the seat 258 of the wheelchair 250, according to an embodiment. The fabric sling 302a, 302b may be configured to wrap around the back 252 or the seat 258 and releasably secure to itself to form a sling around the back 252 or the seat 258 of the wheelchair 250. For example, the fabric sling 302a, 302b may include one or more of buckles, straps, hook and loop material, buttons, zippers, etc., that allow the fabric sling 302a, 302b to releasably secure to itself around the back 252 or the seat 258 of the wheelchair 250. In some embodiments, the fabric sling 302a, 302b may releasably secure directly to the back 252 or the seat 258 of the wheelchair 250 rather than to itself. In any event, the fabric sling 302a, 302b forms a pouch between the fabric sling 302a, 302b and the back 252 or the seat 258, respectively, sized to hold a urine collection container 314 therein.

FIG. 3B shows a urine collection system 300 that may be used with one or more of the fabric slings 302a, 302b. The urine collection system 300 also may be used with other container supports described herein, such as the container supports of urine collection systems 200, 400, 430, 450, 460. The urine collection system 300 may include the urine collection device 212, a urine collection container 314, and the conduit 217a providing a fluid communication between the urine collection device 212 and the urine collection container 314. The orientation of the urine collection system 300 in FIG. 3B demonstrates positioning and orientation of the urine collection container 314 when supported by the fabric sling 302b underneath the seat 258 of the wheelchair 250. This orientation results in the technical effect of allowing urine discharged into the urine collection device 212 to flow downward with gravity through the conduit 217a and into the urine collection container 314.

Although not shown in FIG. 3B, the urine collection system also may include a pump configured to pull urine from the urine collection device 212 and/or push urine in the conduit 217a into the urine collection container 314. The pump may include any aspect of the pump 216 described above. In some embodiments, the urine collection system 300 may include a pump, such as a peristaltic pump, positioned between the urine collection device 212 and the urine collection container 314 that is configured to pull urine from the urine collection device 212 and force the urine into the urine collection container 314. In some embodiments, the pump 216 is not necessary due to the configuration and orientation of the urine collection container 314, and the pump 216 may be absent.

Turning to FIG. 3C, the urine collection container 314 may include an impermeable and flexible layer 355 defining an interior region, an absorptive material 360 positioned in the interior region, and a port or adapter 365 configured to detachably secure to the conduit 217a effective to provide fluid communication between the interior region conduit 217a. The urine collection container 314 also may include a one way valve or filter at the port or adapter 365 resulting in the technical effect of allowing urine to enter the interior region of the urine collection container 314 through the one way valve or filter and preventing urine from exiting the interior region of the urine collection container 314 through the one way valve or filter. With the flexible outer layer 355, the urine collection container 314 may include a urinary drainage bag or have a bag-like characteristics. The urine collection container 314 also may include a semi-rigid plastic that is less rigid than the conduit 217a. In some embodiments, the urine collection container 314 includes a pouch having the flexible layer 355 of a polymer material. The flexible layer 355 may be the outer layer or have other layers positioned outside the flexible layer.

FIG. 3D shows the absorptive material 360 of the urine collection container 314 without the flexible layer 355, according to an embodiment. The absorptive material 360 may provide the technical effect of eliminating or inhibiting leaks in urine collection bags by absorbing the urine, thereby preventing the urine from leaking through any holes or openings in the urine collection bag. The absorptive material also may result in the technical effect of eliminating noise from fluid collection and/or movement (such as a sloshing or splashing noise) common to conventional urine collection systems.

The absorptive material 360 may be removably positioned within the interior region of the urine collection container 314 providing the technical effect of allowing a user or caregiver to insert and/or remove the absorptive material 360. Accordingly, a soiled absorptive material 360 may be removed from the urine collection container 314 and replaced with a fresh absorptive material. In some embodiments, the absorptive material 360 is formed into a shaped having two opposing generally flat or planar surfaces 362 before and/or after absorption of the urine. Having the absorptive material 360 in the urine collection container 314 on the same or a similar plane as the urine flowing into the interior region of the urine collection container 314 (shown in FIG. 3B) may result in the technical effect of increasing or improving absorption of the urine by the absorptive material 360. In some embodiments, the absorptive material 360 may be formed into other shapes, sizes, and configurations. In some embodiments, the absorptive material 360 may line and/or be secured to one or more inner surfaces of the urine collection container 314.

The absorptive material 360 may be configured to expand as urine is absorbed. For example, FIG. 3E shows the absorptive material 360a before urine has been absorbed by the absorptive material 360a and FIG. 3F shows the absorptive material 360b after urine has been absorbed by the absorptive material 360b. As such, the flexible layer 355 of the urine collection container 314 may configured to allow the absorptive material 360 to expand within the urine collection container 314.

In some embodiments, the absorptive material 360 includes a nanomaterial or nanofiber, including a nanofiber membrane. For example, the absorptive material 360 may include an electrospun nanofiber such as cellulose nanofibers and/or electrospun cellulose acetate nanofibers. The nanofibers provide the technical effect of allowing flexibility in manufacturing of different shapes of the absorptive material. The nanofiber of the absorptive material 360 may be woven into fabric that, when hydrated, forms a structured hydrogel. Accordingly, the absorptive material 360 may at least partially solidify urine collected in the urine collection container 314.

An absorptive material 360 including cellulose nanofibers and/or electrospun cellulose acetate nanofibers is not only capable of absorbing large amounts of liquid, but also biodegradable. Conventional diapers, menstrual pads, and other sanitary napkins use super-absorbent polymers that are not biodegradable. The absorptive material 360 including cellulose nanofibers and/or electrospun cellulose acetate nanofibers, then, may provide the technical effect of reducing the environmental impact of using a conventional super-absorbent polymer in a urine collection container. In some embodiments, the absorptive material 360 may include a biodegradable, a non-biodegradable material, or a combination of a biodegradable and a non-biodegradable material.

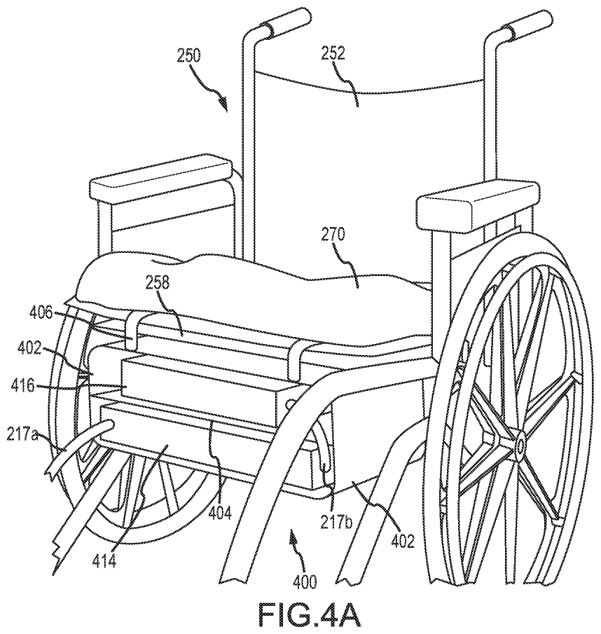

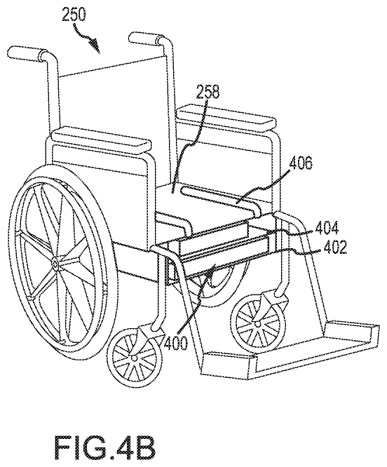

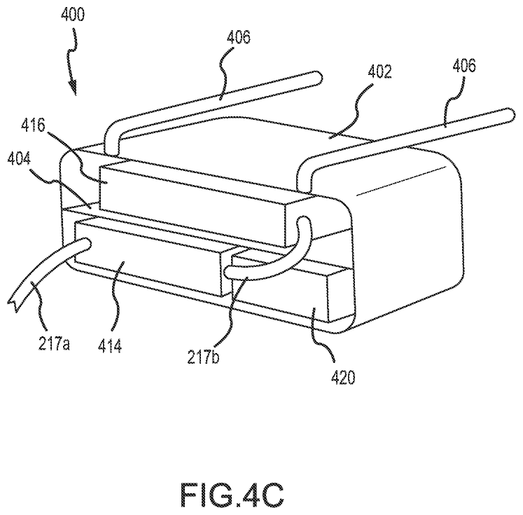

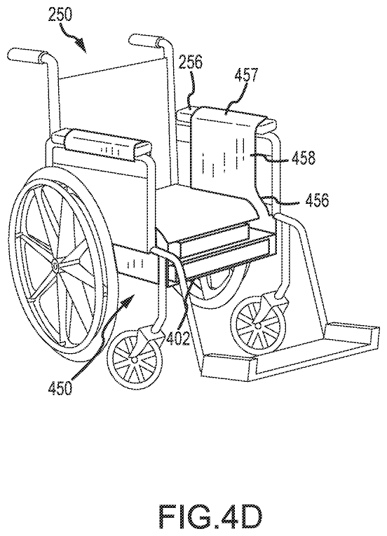

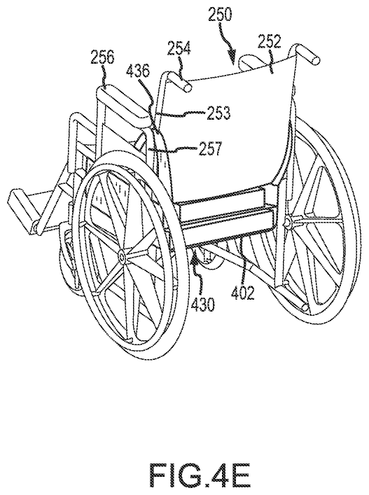

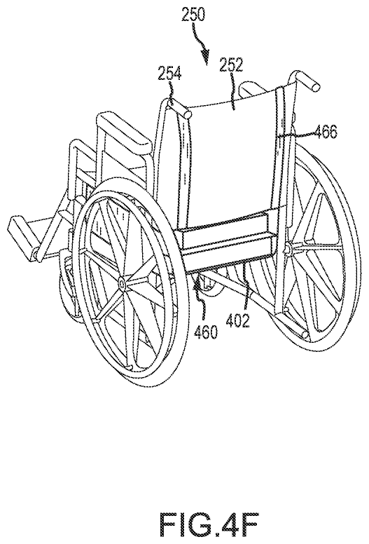

In some embodiments, a container support of a urine collection system may include a compartment mounted to a wheelchair with one or more brackets. FIGS. 4A-4F show various embodiments urine collection systems having a compartment as a container support. The urine collection systems provide the technical effect of allowing the urine collection systems to be mounted to a wide range of chair types with one or more brackets. At least one, some, or all of the embodiments of urine collection systems described herein provide the technical effect of enabling portability of use of the urine collection system in wheelchair units. For example, in skilled nursing facilities, patients can spend significant time during the day in chairs and are ideally monitored for wetting on a 2-hour basis, which 2-hour basis maybe difficult for staff to meet. If a patient is wearing an absorbent product which becomes wetted for an extended period of time, the skin has the potential to break down and result in difficult-to-heal wounds, pain, and requires additional care and follow-up. At least one, some, or all of the embodiments of the urine collection systems described herein provide the technical effect of reducing urine-skin wetting episodes and a portable, wheelchair compatible system enables daytime use of the urine collection device. At least one, some, or all of the embodiments of the portable, discreet urine collection systems described herein may result in the technical effect of improving quality of life for patients using the urine collection system to enable both night and day use and improving the caregiver experience. Embodiments having below chair storage of urine results in the technical effect of enabling ease in discretion or concealment of the urine collection container. The compartments support the urine collection container also result in the technical effect of providing additional options for managing pump sound, such as dampening sound or transferring of pump vibrations to foam or nonwoven liner in the compartment.

Turning specifically to FIGS. 4A and 4B, a urine collection system 400 may include a compartment 402 configured as a container support, one or more brackets 406 secured to the compartment 402 and positioned to mount the compartment 402 to the seat 458 of the wheelchair 250, a pump 416, a urine collection container 414, and the conduit 217a. Unless otherwise noted, the urine collection system 400 may include any aspect of the urine collection systems 200, 300.

The pump 416 may include any aspect of the pump 216 of the urine collection system 200. The pump 416 is in fluid communication with an interior region of the urine collection container 414 such that the pump 416 may pull a vacuum on the interior region of the urine collection container to draw urine from the urine collection device (not shown), through the conduit 217a and into the interior region of the urine collection container 414. The pump 416 may be mounted directly to the urine collection container 414 or fluidly coupled to the urine collection container 414 by the conduit 217a. In some embodiments, the pump 416 may include an in-line component such as a filter or condensation trap configured to prevent or inhibit condensation from transferring to the pump.

The urine collection container 414 may include any aspect of the urine collection containers 214, 314. For example, the urine collection container 414 may be generally rigid. In some embodiments, the urine collection container 414 may include a flexible layer and have an absorbent material 360 positioned therein. At least one (e.g. both) of the pump 416 and the urine collection container 414 may have a generally flat or planar profile to fit within the compartment 404. The interior region of the urine collection container 414 may hold about 0.5 L to about 3 L of urine, about 1 L to about 2 L of urine, about 0.5 L of urine, about 1 L of urine, about 1.2 L of urine, about 1.5 L of urine, about 2 L of urine, about 2.5 L of urine, or about 3 L of urine.

In some embodiments, the urine collection container 414 includes of a form factor compatible with one or more sections of the urine collection container being expandable or collapsible to aid in filling and/or emptying the urine collection container 414. The urine collection container 414 may be compatible with efficient emptying, cleaning, and reuse. In some embodiments, the urine collection container 414 may include an expandable bladder or bag, bellows, or a replaceable pouch. The urine collection container 414 also may include an exit port or door for emptying the urine collection container 414, such as a stop cock, a sliding valve, a hinged door, a toggle catch, a sliding latch, a push button release, and/or a lid. In some embodiments, the urine collection container 414 is sealed through interference fit or gaskets.

The compartment 402 may be sized and dimensioned to hold or support the urine collection container 414 and the pump 416 therein. The urine collection container 414 and the pump 416 may be removable and replaceable within the compartment 402. The compartment 402 may include an opening positioned to allow insertion, removal, and/or viewing of the pump 416 and the urine collection container 414. The urine collection system 400 may be configured such that the opening of the compartment 402 is oriented at the front of the wheelchair 250 when the compartment 402 is mounted to the wheelchair 250, according to an embodiment. In some embodiments, the urine collection system 400 may be configured such that the opening of the compartment 402 is oriented at the back of the wheelchair 250. In some embodiments, the compartment 402 includes one or more walls, such as a top wall, a bottom wall, two opposing side walls, and a rear wall distal to the opening. The compartment 402 may be generally rigid and/or formed to maintain its shape when mounted to the wheelchair 250 and supporting at least the urine collection container 414 and the pump 416. For example, the compartment 402 may be include one or more of a rigid frame and/or at least one rigid wall. The rigid material of the frame and/or the wall may include plastic, metal, wood, carbon fiber, and/or any other material configured to maintain its shape when the compartment 402 is supporting at least the pump 416 and the urine collection container 414. As the compartment 402 is configured to be removably mounted to the wheelchair 250, the compartment may be transferred or moved to other positions, such as on a bedside table, without removing or adjusting the pump 416 and the urine collection container 418 in the compartment 402.

In some embodiments, the compartment 402 includes at least one shelf 404 positioned or positionable therein. The shelf 404 may be removably and/or adjustably mounted within compartment 404. When the shelf 404 is inserted or positioned in the compartment 402, the compartment 402 includes at least two regions that may be used as shelves to support the urine collection container 414 and/or the pump 416: the bottom wall of the compartment 402 and the shelf 404. The shelf 404 may be positioned to support at least one of the pump 416 and/or the urine collection container 414 above the shelf 404 when the compartment is mounted to the wheelchair 250. For example, the shelf 404 may be positioned to support the pump 416 between the shelf 404 and the top wall of the compartment 402, with the urine collection container 414 being positioned between the shelf 404 and the bottom wall of the compartment 402. In some embodiments, the shelf 404 may be positioned to support the urine collection container 414 between the shelf 404 and the top wall of the compartment 402, with the pump 416 being positioned between the shelf 404 and the bottom wall of the compartment 402. The compartment 402 and/or the shelf 404 provide easy access to connections between the conduits 217a, 217b and the pump 416 and the urine collection container 414. In some embodiments, the compartment 402 also includes a foam and/or nonwoven liner in the compartment that dampens sounds or inhibits transfer of pump vibrations.

The brackets 406 may be secured to a top wall and/or side of the compartment 402. In some embodiments, the brackets 406 are secured to the compartment to fit the seat 258 between a portion of the bracket 406 and the top wall of the compartment 402. In some embodiments, the brackets 406 may be generally U-shaped, having a first arm secured to the top wall of the compartment 402, a second arm positioned to hang from the seat 258 of the wheelchair 250, and a central portion extending between the first arm and the second arm. The seat 258, then, may slide between the two arms of the brackets 406 to hang or mount the compartment 402 from the seat. In some embodiments, the brackets 406 are adjustable on the compartment 402 to increase or decrease a distance between the brackets 406. In some embodiments, the brackets 406 are adjustable to increase or decrease a length of the portion of the brackets 406 positioned to contact or hang from the seat 258 of the wheelchair 250. In some embodiments, the brackets 406 are adjustable to increase or decrease a distance between the portion of the brackets 406 positioned to contact or hang from the seat 258 and the compartment 402 such that the urine collection system 400 can be adapted for different wheelchairs.

A cushion 270 may be positioned over the seat 258 and the brackets 406. In some embodiments, the cushion 270 may cover or including routing for at least a portion of the conduit 217a resulting in the technical effect of making the urine collection system 400 more discrete. In some embodiments, the cushion 270 or other seating elements may be secured to a portion of the brackets 406 positioned to hang from the seat 258. The cushion 270 or other seating element secured to the portion of the bracket 406 positioned to hang from the seat 258 also may include an additional secondary absorbent sheet secured or securable thereto. In some embodiments, the urine collection system 400 may include a sleeve secured to the arm 256 of the wheelchair 250 that includes routing for the conduit 217a.

In some embodiments, the urine collection system 400 may include a sensor and/or a controller. The sensor of the urine collection system 400 may include any aspect of the sensor 215 and the controller may include any aspect of the controllers 221, 500. For example, the sensor and the controller of the urine collection system 400 may be configured to enable monitoring of fluid volume in the urine collection container 414 via the sensor and notification via the controller of when the urine collection container 414 requires emptying. The sensor and/or the controller of the urine collection system 400 also may be configured to enable monitoring of the flow rate of urine into the urine collection container 414, monitoring of a vacuum function in the urine collection system 400, and monitoring of the volume of urine in the urine collection container 414.

In some embodiments, the urine collection system 400 includes a display on at least one of the pump 416, the urine collection container 414, or an additional electronic device. In some embodiments, the controller of the urine collection system 400 is configured to wirelessly communicate with an electronic device of the user or caregiver having a display. The urine electronic device may be secured to the wheelchair. The urine collection system 400 may be configured to coordinate a visual indication of the volume of urine in the urine collection container 414 on the display of any of pump 416, the urine collection container 414, the additional electronic device, or the electronic device of the user or caregiver. The controller of the urine collection system 400 also may be configured to communicate with an application on electronic device configured to provide notifications of one or more statuses of the urine collection system 400, such as information relating usage history of the urine collection container 414 and/or the pump 416. In some embodiments, the urine collection system 400 may include an alert electronic device configured to wirelessly communicate with the controller and secured to the wheelchair 250, such as below the arm 256 of the wheelchair 250. The alert electronic device may be configured to provide an audible, visual, or haptic feedback alert when emptying of the urine collection device 414 is suggested.

Turning to FIG. 4C, in some embodiments, the urine collection system 400 includes a cleaning module 420 sized and dimensioned to be housed in the compartment 402 with the pump 416 and/or the urine collection container 414. For example, the cleaning module 420 may be positioned next to one of the urine collection container 414 or the pump 416. The cleaning module 420 may include a cleaning solution housed therein, such as an alcohol or vinegar based solution. The cleaning module 420 also may include a port configured to secure to the conduit 217a effective to provide fluid communication between the cleaning module 420 and the interior region of the urine collection container 414. In operation, activating the pump 416 may pull a vacuum on the urine collection container 414 effective to pull cleaning solution from the cleaning module 420 through the conduit 217a and into the interior region of the urine collection container 414. This configuration provides the technical effect of allowing quick and efficient cleaning of the conduit 217a and/or the urine collection container 414.

Turning to FIG. 4D, in some embodiments, a urine collection system 450 may include brackets 456 or other elements configured to mount or hang from the arms 256 of the wheelchair 250. Unless otherwise noted, the urine collection system 450 may include any aspect of the urine collection system 400, such as the compartment 402, the urine collection container 414, the pump 416, the sensor, the conduits 217a, 217b, and/or the shelf 404.

The one or more brackets 456 or other elements include a portion or region 457 positioned to mount over and/or hang from the arm 256 of the wheelchair 250. In some embodiments, at least the region 457 positioned to mount over the arm 256 is generally rigid or configured to wrap around the arm 256 to be releasably secured around the arm 256. An intermediate portion or region 458 may be generally rigid and may extend between the compartment 402 and the region 457 of the bracket 456. In some embodiments, the intermediate region 458 of each of the brackets 456 is positioned between the arms 256 of the wheelchair 250, as shown in FIG. 4D. In some embodiments, the intermediate region 458 of each of the brackets 456 is positioned outside the arms 256 rather than between the arms 256 of the wheelchair 250. A region of the bracket 456 distal to the region 457 may be secured to the compartment 402 effective to support the compartment 402 below the seat 458 when the region 457 is mounted to and/or hanging from the arm 256.

Turning to FIGS. 4E and 4F, in some embodiments, a urine collection system may include the compartment 402 oriented to have the opening at the back of the wheelchair 250 when the compartment 402 is mounted to the wheelchair 250. With specific reference to FIG. 4E, in some embodiments, a urine collection system 430 may include brackets 436 or other elements configured to mount or hang from the rear region of the wheelchair 250. Unless otherwise noted, the urine collection system 430 may include any aspect of the urine collection system 400, such as the compartment 402, the urine collection container 414, the pump 416, the sensor, the conduits 217a, 217b, and/or the shelf 404.

The brackets 436 of the urine collection system are secured or securable to the compartment 402 and configured to mount to or hang from one or more an arm frame member 257 or a back frame member 253 of the wheelchair 250. In some embodiments, each bracket 436 is configured to hang from the frame of the wheelchair 250, such as an intersection between the arm frame member 257 and the back frame member 253. The bracket 436 also may wrap around the back 252 of the wheelchair 250, according to an embodiment.

With specific reference to FIG. 4F, in some embodiments, a urine collection system 460 may include brackets 466 or other elements configured to mount or hang from the back 252 of the wheelchair 250. Unless otherwise noted, the urine collection system 460 may include any aspect of the urine collection system 400, such as the compartment 402, the urine collection container 414, the pump 416, the sensor, the conduits 217a, 217b, and/or the shelf 404. The brackets 466 of the urine collection system are secured or securable to the compartment 402 and configured to mount to or hang from the back 452 of the wheelchair 250.

FIG. 5 is a schematic of a controller 500 that may be used with any of the systems and methods described herein, according to an embodiment. For example, the controller 221 may include any aspect of the controller 500. The controller 500 may be configured to implement any of the example acts or steps disclosed herein, such as wired or wirelessly communicating with the sensor 215 or other electronic devices, and/or determining a volume of the urine 55 in the urine collection container 214, 314, 414.

The controller 500 includes at least one computing device 510, according to an embodiment. The at least one computing device 510 is an exemplary computing device that may be configured to perform one or more of the acts described above. The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512. In some examples, the at least one computing device 510 may include a plurality of computing devices. Components of computing device 510 shown in FIG. 5 are described in additional detail below.

In some examples, the processor(s) 520 includes hardware for executing instructions (e.g., instructions for carrying out one or more portions of any of the methods disclosed herein), such as those making up a computer program. For example, to execute instructions, the processor(s) 520 may retrieve (or fetch) the instructions from an internal register, an internal cache, the memory 530, or a storage device 540 and decode and execute them. In particular examples, processor(s) 520 may include one or more internal caches for data such as tables pertaining to volumetric tables. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods disclosed herein.

In some examples, the processor 520 is configured to perform any of the acts disclosed herein or cause one or more portions of the computing device 510 or controller 500 to perform at least one of the acts disclosed herein. Such configuration can include one or more operational programs (e.g., computer program products) that are executable by the at least one processor 520. For example, the processor 520 may be configured to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a change of filter is suggested, and/or automatically transmit an alert when a change or recharge of battery is suggested.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, one or more of a history of the volume of the urine in the urine collection container, a trend of the volume of the urine in the urine collection container, a history of filter replacement, and/or a history of battery replacement or recharging may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted access the data in the memory storage medium(s) to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a change of filter is suggested, and/or automatically transmit an alert when a change or recharge of battery is suggested. For example, the at least one processor 520 may access one or more look-up tables in the memory storage medium(s) such as memory 530 or storage device 540.

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512 or one or more networks. For example, communication interface 560 may include a network interface controller (NIC) or network adapter for communicating with an Ethernet or other wire-based network or a wireless NIC (WNIC) or wireless adapter for communicating with a wireless network, such as a WI-FI. The one or more additional computer device 512 may include a smart phone of the user, a smart phone of the caregiver, an electronic device on the wheelchair, and/or a computer device of a healthcare system.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. The computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

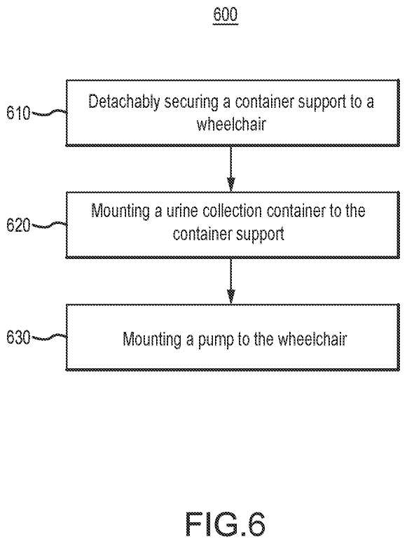

FIG. 6 is a flow diagram of a method 600 for assembling a portable urine collection system, according to an embodiment. The method 600 includes an act 610 of detachably securing a container support to a wheelchair. The method 600 also includes an act 620 of mounting a urine collection container to the container support. The urine collection container may include a sensor secured thereto and configured to detect a property relating at least to a volume of the urine in the urine collection container. In some embodiments, the method 600 also may include positioning a urine collection device proximate to a urethra of a user and fluidly coupling the urine collection device to the urine collection container with a first conduit. The method also includes an act 630 of mounting a pump to the wheelchair. The act 630 may include mounting a pump to the wheelchair with the pump in fluid communication with the urine collection container and configured to pull a vacuum on the interior region of the urine collection container effective to draw urine from the urine collection device through the first conduit into the urine collection container.

The method 600 may include assembling any of the urine collection systems described herein. For example, the act 610 may include securing one or more straps of a pack to one or more handles of the wheelchair. In some embodiments, the act 620 of mounting a urine collection container to the container support includes positioning the urine collection container inside the pack to mount the urine collection container. The method 600 also may include an act of positioning the urine collection container inside the pack to mount the urine collection container includes positioning the urine collection container inside a sleeve inside the pack. In some embodiments, the method 600 further includes an act of fluidly coupling the urine collection container to the pump with a second conduit and/or securing the first conduit to a user or the wheelchair with a belt.

In some embodiments, the act 610 of detachably securing a container support to a wheelchair may include mounting a compartment of the container support to the wheelchair with one or more brackets secured to the compartment. In some embodiments, mounting a compartment of the container support to the wheelchair with one or more brackets secured to the compartment includes mounting or hanging a portion of the one or more brackets from a seat of the wheelchair with the seat positioned between the portion of the one or more brackets and the compartment. In some embodiments, mounting a compartment of the container support to the wheelchair with one or more brackets secured to the compartment includes mounting a portion of the one or more brackets over one or more arms of the wheelchair to position the compartment below a seat of the wheelchair. In some embodiments, mounting a compartment of the container support to the wheelchair with one or more brackets secured to the compartment includes mounting a portion of the one or more brackets to a back of the wheelchair with the compartment positioned below a seat of the wheelchair. The act 620 of mounting a urine collection container to the container support may include positioning the urine collection container on a shelf in the compartment.

In some embodiments, the act 610 of detachably securing a container support to a wheelchair may include detachably securing a sling one or more of a back or a seat of the wheelchair. The method 600 may include an act of inserting a nanofiber membrane into the urine collection container, the nanofiber membrane including electrospun cellulose acetate nanofibers configured to expand as urine is absorbed by the nanofiber membrane in the urine collection container. The act 620 of mounting a urine collection container to the container support may include mounting a urine collection container to the container support includes positioning the urine collection container between the sling and the back or the seat of the wheelchair.

The acts of the method 600 described above are for illustrative purposes. For example, the acts of the method 600 can be performed in different orders, split into multiple acts, modified, supplemented, or combined. In an embodiment, one or more of the act of the method 600 can be omitted from the method 600. Any of the acts of the method 600 can include using any of the portable urine collection systems disclosed herein.

A method of collecting fluids from a user is described, according to an embodiment. The method may include any of the fluid collection systems described herein. In an embodiment, the method includes fluidly coupling a conduit to a urine collection container. Fluidly coupling a conduit to a urine collection container may include fluidly coupling the conduit to a port on the urine collection container. The method of collecting fluids from a user may further include allowing, with a one way valve, the urine to pass from the conduit into the interior region of the urine collection container and preventing, with the one way valve, the urine from passing from the interior region of the urine collection container out of the port.

In some embodiments, the method of collecting fluids from a user also may include positioning a urine collection device proximate to a urethra of a user with the conduit providing fluid communication between the urine collection device and an interior region of the urine collection container. The method also may include mounting the urine collection container to a wheelchair with a sling.

In some embodiments, the method of collecting fluids from a user also may include absorbing urine with a urine absorption material positioned in the interior region of the urine collection container after the urine has been discharged by the user into the urine collection device and passed through the conduit into the interior region of the urine collection container. The absorptive material may be removable and/or replaceable. The method, therefore, may include inserting the absorptive material into interior region of the urine collection container. The urine absorption material includes a nanofiber membrane, such as electrospun cellulose acetate nanofibers, according to an embodiment. The nanofiber membrane may form a structured hydrogel when hydrated with the urine from the urine collection device. The urine absorption material expands as the urine is absorbed into the urine absorption material. In some embodiments, the method also includes pumping the urine into the urine collection container.

The acts of the method of collecting fluids from a user described above are for illustrative purposes. For example, the acts of the method of collecting fluids from a user can be performed in different orders, split into multiple acts, modified, supplemented, or combined. In an embodiment, one or more of the acts of the method of collecting fluids from a user can be omitted from the method. Any of the acts of the method of collecting fluids from a user can include using any of the portable urine collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A portable urine collection system, comprising:
a urine collection device configured to be positioned at least proximate to a urethra of a user;
a first conduit in fluid communication with the urine collection device;
a urine collection container having an interior region;
a nanofiber membrane positioned or positionable within the urine collection container and configured to absorb the urine entering the urine collection container from the urine collection device; and
a container support configured to detachably secure to a wheelchair and support at least the urine collection container.

2. The portable urine collection system of claim 1, further comprising a pump in fluid communication with the urine collection container and configured to pull a vacuum on the first conduit effective to draw urine from the urine collection device through the first conduit into the urine collection container.

3. (canceled)

4. The portable urine collection system of claim 2, further comprising a power source operably coupled to the pump.

5. The portable urine collection system of claim 2, wherein the pump includes an exhaust filter configured to at least partially filter air exhausted from the pump and an aromatherapy accessory secured or securable to the pump proximate to the exhaust filter.

6. (canceled)

7. The portable urine collection system of claim 1, further comprising:
a sensor secured or securable to the container and configured to detect a property relating at least to a volume of the urine in the urine collection container; and
a controller configured to communicate with the sensor and wirelessly transmit an alert to an electronic device when the property relating at least to the volume of the urine detected by the sensor indicates the volume of the urine in the urine collection container has reached or exceeded a predetermined volume.

8. (canceled)

9. The portable urine collection system of claim 2, wherein the container support includes a pack having one or more straps securable to one or more handles of the wheelchair, the pack being sized and dimensioned to hold at least the urine collection container, the sensor, and the pump therein.

10. The portable urine collection system of claim 9, further comprising a second conduit positioned between the urine collection container and the pump, the second conduit providing fluid communication between the pump and the interior region of the urine collection container.

11. The portable urine collection system of claim 9, further comprising one or more sleeves in the pack sized and dimensioned to hold at least a portion of the urine collection container and the pump within the pack.

12. The portable urine collection system of claim 11, further comprising one or more foam members positioned or positionable on at least one sleeve of the one or more sleeves effective to reduce operational sounds of the pump.

13. The portable urine collection system of claim 9, wherein at least portion of the first conduit is at least partially opaque.

14. The portable urine collection system of claim 9, wherein the sensor includes a level transmitter configured to detect a level of the urine in the urine collection container and an accelerometer configured to determine when the level transmitter detects the level of the urine in the urine collection container.

15. The portable urine collection system of claim 9, further comprising a belt configured to secure the first conduit to a user or the wheelchair.

16. The portable urine collection system of claim 2, wherein the container support includes a compartment and one or more brackets secured or securable to the compartment and positioned or positionable to detachably mount to the wheelchair, the compartment having a first shelf sized and dimensioned to hold the pump thereon and a second shelf sized and dimensioned to hold the urine collection container thereon.

17. The portable urine collection system of claim 16, wherein the one or more brackets include a portion positioned or positionable to hang the compartment from a seat of the wheelchair with the seat positioned between the portion of the one or more brackets and the compartment.

18. The portable urine collection system of claim 16, wherein the one or more brackets include a portion shaped to mount over one or more arms of the wheelchair to position the compartment below a seat of the wheelchair when the portion of the one or more brackets is mounted to the one or more arms of the wheelchair.

19. The portable urine collection system of claim 16, wherein the one or more brackets include a portion shaped to mount to a back of the wheelchair with the compartment positioned below a seat of the wheelchair when the one or more brackets are mounted on the back of the wheelchair.

20. (canceled)

21. The portable urine collection system of claim 1, wherein the nanofiber membrane includes electrospun cellulose acetate nanofibers.

22. The portable urine collection system of claim 1, wherein the container support includes a fabric sling configured to detachably secure to one or more of a back or a seat of the wheelchair.

23-33. (canceled)

34. A urine collection system, comprising:
a urine collection device configured to be positioned at least proximate to a urethra of a user;
a urine collection container having an interior region;
a urine absorption material positioned within the urine collection container, the urine absorption material including a nanofiber membrane; and
a conduit in fluid communication with the urine collection device and the urine collection container.

35. (canceled)

36. The urine collection system of claim 34, wherein the nanofiber membrane includes electrospun cellulose acetate nanofibers.

37. The urine collection system of claim 34, wherein the nanofiber membrane forms a structured hydrogel when hydrated with the urine from the urine collection device.

38. (canceled)

39. The urine collection system of claim 34, wherein the urine absorption material is shaped generally complementary to a shape of the urine collection container.

40. The urine collection system of claim 34, wherein the urine absorption material is removable from the urine collection container.

41. The urine collection system of claim 34, wherein the urine absorption material is secured to and lines one or more inner surfaces of the urine collection container.

42. The urine collection system of claim 34, wherein the urine absorption material is substantially planar.

43. The urine collection system of claim 34, wherein the urine collection container includes a polymer pouch.

44. The urine collection system of claim 34, wherein the urine collection container includes a port configured to secure to the conduit and a one-way valve configured to allow urine to pass from the conduit into the interior region of the urine collection container and prevent urine from passing from the interior region of the urine collection container out of the port.

45. The urine collection system of claim 34, further comprising a pump positioned between urine collection device and the urine collection container, the pump configured to draw urine from the urine collection device into the conduit and/or push urine in the conduit into the urine collection device.

46. The urine collection system of claim 34, further comprising a sling configured to detachably secure to at least one of a back or a seat of a wheelchair and mount the urine collection container to the wheelchair.

47. A method of collecting fluids from a user, the method comprising:
fluidly coupling a conduit to a urine collection container;
positioning a urine collection device proximate to a urethra of a user with the conduit providing fluid communication between the urine collection device and an interior region of the urine collection container; and
absorbing urine with a urine absorption material positioned in the interior region of the urine collection container after the urine has been discharged by the user into the urine collection device and passed through the conduit into the interior region of the urine collection container, wherein the urine absorption material includes a nanofiber membrane.

48. (canceled)

49. The method of claim 47, further comprising inserting the absorptive material into interior region of the urine collection container.

50. (canceled)

51. The method of claim 47, wherein the nanofiber membrane includes electrospun cellulose acetate nanofibers.

52. The method of claim 47, wherein the nanofiber membrane forms a structured hydrogel when hydrated with the urine from the urine collection device.

53. (canceled)

54. (canceled)

55. (canceled)

56. The method of claim 47, further comprising mounting the urine collection container to a wheelchair with a sling.
